(19) United States
(12) Patent Application Publication
(10) Pub. No.: US 2016/0060542 A1
(43) Pub. Date: Mar. 3, 2016

(54) FLUIDIZED BED UNIT STARTUP

(71) Applicants: Masaaki SUGITA, The Woodlands, TX (US); Christopher Gordon SMALLEY, Manassas, VA (US); George Phillip CHARLES, Centreville, VA (US); Robert Gerard TINGER, Friendswood, TX (US); Terrance Charles OSBY, Manvel, TX (US); Allen Scott GAWLIK, Houston, TX (US)

(72) Inventors: Masaaki SUGITA, The Woodlands, TX (US); Christopher Gordon SMALLEY, Manassas, VA (US); George Phillip CHARLES, Centreville, VA (US); Robert Gerard TINGER, Friendswood, TX (US); Terrance Charles OSBY, Manvel, TX (US); Allen Scott GAWLIK, Houston, TX (US)

(73) Assignee: ExxonMobil Research and Engineering Company, Annandale, NJ (US)

(21) Appl. No.: 14/827,505

(22) Filed: Aug. 17, 2015

Related U.S. Application Data
(60) Provisional application No. 62/041,882, filed on Aug. 26, 2014.

(51) Int. Cl. C10G 11/20; C07C 2/86; C10G 11/05; C07C 1/20

(52) U.S. Cl. CPC C10G 11/20; C07C 1/20; C07C 2/864; C10G 11/05; C07C 2529/40

(57) ABSTRACT

The startup of a fluidized bed process unit uses an air heater to raise the temperature of the unit to the level necessary for operation of the unit to be self-sustaining in its normal operating regime without the use of torch oil. This startup sequence is particularly useful for fluidized bed units which utilize a circulating catalyst with particular emphasis on endothermic conversion units such as FCC and Resid Catalytic Cracking (RCC), but also on other catalytic units with circulating catalyst inventories such as various exothermic conversion, e.g. methanol conversion, processes. Elimination of the torch oil injection enables catalyst selectivity/activity to be retained during startup and at any other time that the heat requirement of the unit cannot be met by the internal functioning of the process, e.g. by coke generation during the reaction and combustion during regeneration of the catalysts or during the reaction itself.

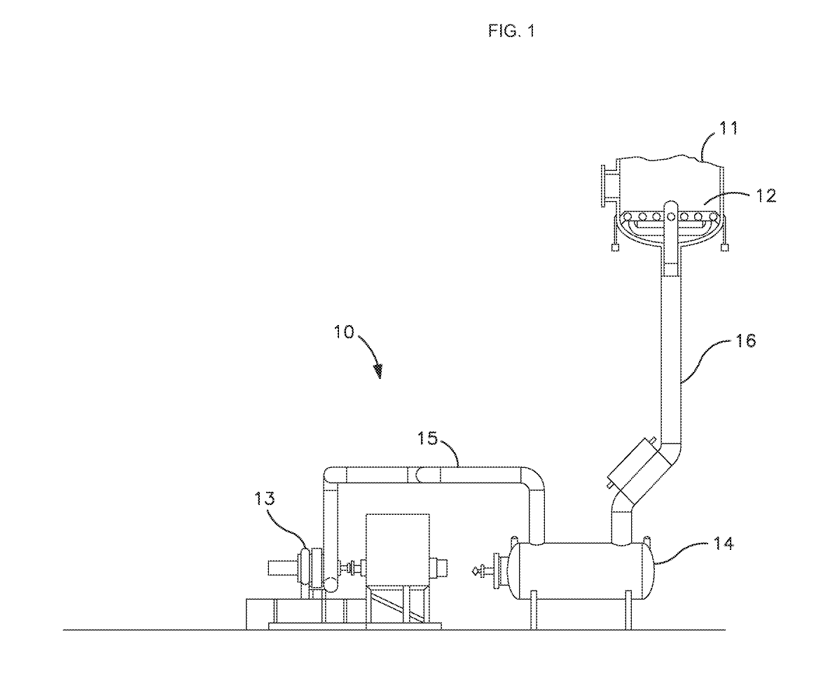

This application claims priority to U.S. Provisional Application Ser. No. 62/041,882 filed Aug. 26, 2014, herein incorporated by reference in its entirety.

### FIELD OF THE INVENTION

This invention relates to a procedure for starting fluidized bed process units which need to be started at an elevated temperature.

### BACKGROUND OF THE INVENTION

The initial industrial application of fluidization took place in a reactor for coal gasification process. The use of circulating fluid bed processes began in the early 1940s with the development by Esso of the Fluid Catalytic Cracking (FCC) process for heavy oil conversion, the basic principles of which were later extended to other processes, both catalytic and non-catalytic. The fluidized bed technique is notable for its capability of promoting high levels of contact between gases and solids with excellent heat transfer between the solid and fluid phases. As such, the technique is in widespread use for the purpose of regulating the temperature of reactions where heat generation or consumption is a problem, either in requiring large quantities of heat to be removed or delivered. Reactions where it is desirable to remove a by-product of the reaction quickly, for example, water, are also suitable for fluidized bed operation in view of the highly effective mass transfer which may take place in the bed.

Fluidized bed units are particularly well suited to use with processes which require continuous circulation of a finely divided particulate material between one zone and another where the two zones have some differing characteristic, for example, of temperature or atmosphere. Many fluidized process units, notably the FCC units, are used with processes in which a catalytic material is required to pass from a reaction zone to a regeneration zone at a different, usually higher, temperature; the process is particularly appropriate in cases in which the catalytic material becomes inactivated by the deposition of carbon as a by-product of the reaction with this carbon being removed by oxidative combustion in a regenerator at a higher temperature. In the petroleum refining and petrochemical industries, a number of such processes are to be found, including FCC and its variant, Resid Fluid Catalytic Cracking (RF?CC), Toluene Alkylation including Ethylation to MEB (methylethylbenzene), Benzene and/or Toluene Methylation with Methanol or dimethyl ether (DME) to Aromatics such as Paraxylene, Benzene Ethylation to DEB (Diethylbenzene) as well as a number of methanol conversion processes including Methanol to olefins (MTO). Methanol to aromatics (MTA). Methanol to Paraxylene (MTPX), Methanol to Gasoline (MTG), Syngas to Olefins. Catalytic processes such as these invariably require a startup procedure to be followed in which the reactor and, if present, the regenerator, are initially raised to an elevated temperature, sometimes as high as about 500-600° C. as in FCC in order for the overall reaction sequence, i.e. the conversion reaction and the regeneration, to take place and become self-sustaining. Startup of these units may also require an initial hot drying step to remove moisture from the refractory lining on the vessels internal. The heat up process is typically done initially by hot air or hot steam to the selected temperature, then introducing burning agent to the regenerator to increase the catalyst temperature in the regenerator and with a start to shifting the hot catalyst to the reactor side. Fluidized bed processes needing to be started at an elevated temperature which may be cited include Syngas to Aromatics. Syngas to Paraxylene, Biomass to Olefins, Biomass to Aromatics, Biomass to Gasoline.

Possibly the most typical startup procedure is to be found with the FCC unit which, as noted above, requires temperatures in the range of 500-600° C. in the regenerator in order to bring hot catalyst to the reactor. A typical startup procedure may commence with a dry-out of the unit by blowing hot air from an air heater into the regenerator and sometimes into the reactor. During this step, the temperature of the regenerator will be increased to a value in the range of ambient to about 100-250° C. The air heater is typically a heat fuelled by gas or oil with the combustion gases fed into the regenerator which may be isolated from the reactor during this time by closing the isolation valves in the catalyst lines connecting the reactor to the regenerator. When the regenerator reaches a certain temperature, the reactor may also be dried by opening the slide valves to permit the hot air to flow into the reactor before steam is introduced by way of the lift injectors at the foot of the riser. Normally, the drying/pre-heating should be continued until the reactor is hot enough to preclude condensation of the steam. When the regenerator and reactor have reached a sufficient temperature, the catalyst can be introduced into the regenerator from the catalyst hopper, followed by the start of catalyst circulation.

The unit heating after loading catalyst can be carried out by burning torch oil in the regenerator. Spray injection nozzles for the oil, normally LCO or gas oil, are provided around the lower periphery of the regenerator in the area where a fluidized dense bed of catalyst is found in operation. Torch oil combustion may be continued when the catalyst is loaded and circulation is started; in some unit torch oil burning may be used continuously or intermittently in order to maintain the required operating temperature in the regenerator, for instance, when insufficient coke is being produced in the reactor; this condition may also be encountered during shut down to control the rate of unit cooling or when the feed supply is interrupted and catalyst circulation continues.

While the use of torch oil in this way is generally considered necessary, it is not without its own problems. It is essential to ensure that the oil lights off properly when injected. Torch oil that does not ignite at the injection points in the dense bed will pass into the upper zones of the vessel, i.e. dilute phase, cyclones, and overhead system, where it can ignite, possibly even explode, with obvious undesirable consequences. For this reason, the temperature of the catalyst dense bed should be safely above the ignition temperature of the oil before it is injected. In addition, there should be an adequate depth of catalyst above the injection nozzles, to ensure proper ignition of the oil and efficient dispersion of the heat into the catalyst bed. Another problem encountered with the use of torch oil is that a higher amount of carbon monoxide is likely to be released into the atmosphere during startup in units without a CO boiler/furnace.

Even if well controlled, the combustion of the torch oil is attended by a deactivation of the catalyst and/or loss of catalyst selectivity. Thermal, hydrothermal and chemical deactivation may occur. Thermal deactivation may result from sintering of the catalyst in the direct region where the oil is combusted and hydrothermal deactivation from the effects of the steam produced in the combustion process on zeolite catalysts which may undergo dealumination and consequent loss of activity and/or selectivity. Chemical deactivation may be encountered when oils such as gas oil or LCO with high sulfur and/or metal contents are used, producing combustion products, e.g. sulfur oxides or vanadium pentoxide which react deleteriously with the zeolite; the torch oil combustion products may also have a negative effect on the process itself in cases where continued torch oil use is required or where the combustion products from the torch oil may be corrosive to the metallurgy of the equipment. While the issue of chemically-induced catalyst deactivation may be reduced by the use of better quality torch oils such as hydrotreated distillates, the possibility of thermal and hydrothermal deactivation/selectivity loss may persist.

There is therefore a need to minimize or eliminate the use of torch oil in fluidized bed process units using catalysts susceptible to deactivation by torch oil combustion.

### SUMMARY OF THE INVENTION

According to the present invention we propose to conduct the startup of a fluidized bed process unit using a separate heater to raise the temperature of the unit to the level necessary for operation of the unit to be self-sustaining in its normal operating regime without the use of torch oil. This startup sequence is particularly useful for fluidized bed units which utilize a circulating catalyst with particular emphasis on FCC and Resid Catalytic Cracking (RCC), but also on other catalytic units with circulating catalyst inventories such as methanol conversion units used for toluene alkylation including ethylation to MEB (methylethylbenzene), benzene and/or toluene methylation with methanol or dimethyl ether (DME) to aromatics such as paraxylene, benzene ethylation to DEB (diethylbenzene) as well as a number of methanol conversion processes including methanol to olefins (MTO), methanol to aromatics (MTA), methanol to paraxylene (MTPX) and methanol to gasoline (MTG).

Elimination of the torch oil injection enables catalyst selectivity/activity to be retained during startup and at any other time that the heat requirement of the unit cannot be met by the internal functioning of the process, e.g. by coke generation during the reaction and combustion during regeneration: catalysts. It also assists in minimizing CO₂ release to the atmosphere for units with CO combustion equipment.

As applied to the Fluid Catalytic Cracking (FCC) process, the FCCU will have a startup in which the unit is heated to a self-sustaining reaction temperature exclusively with heated air from an air heater. The catalytic cracking process itself is one in which a heavy oil feed is cracked in the reactor section of the unit, typically a riser reactor, with a stream of hot cracking catalyst from the regenerator in which the catalyst is regenerated by combustion of coke which accumulates on the catalyst during the cracking of the feed. In the startup procedure, the air heater feeds heated air to the regenerator until the regenerator attains a temperature at which heat from the exothermic combustion of the coke accumulated on the catalyst during the cracking of the heavy oil feed is sufficient to sustain the cracking reaction. Generally, the catalyst will be retained in the catalyst hopper until the regenerator attains the desired temperature for the entire cracking-regeneration cycle to take off on its own as sufficient coke is accumulated on the catalyst to provide the heat from the exothermic combustion of the coke to the extent necessary to maintain the endothermic cracking reaction.

In its application to the methanol conversion process, the process unit itself, like the FCCU, comprises a conversion reactor in which the feed is converted in the presence of a stream of catalyst from the catalyst regenerator in which the catalyst is regenerated by combustion of the coke which accumulates on the catalyst during the conversion of the methanol/DME feed. In the methanol conversion process, a feed stream comprising methanol and/or dimethyl ether (DME) is converted in a fluidized bed methanol conversion process unit at an elevated temperature; the startup of the unit is accomplished by initially heating the reactor and/or the regenerator of the unit with heated fluid from the heater and then the regenerator and the reactor until the unit attains a temperature at which the regenerated catalyst (from which coke accumulated on the catalyst during the conversion) has been combusted in the regenerator has sufficient conversion activity for the feed to sustain the conversion reaction. The catalyst is generally loaded from the catalyst hopper into the regenerator when the regenerator attains the temperature at which the regenerated catalyst has sufficient conversion activity for the conversion reaction.

### DRAWINGS

The single FIGURE of the accompanying drawings is a simplified unit schematic of an air heater configuration for the startup of an FCCU.

### DETAILED DESCRIPTION

The exact process which is to be carried out hi the process unit is not in itself, an important factor: the invention resides in the manner in which the process unit is brought to a temperature at which the reaction can be regarded as self-sustaining, that is, of being carried on indefinitely according to its normal operating regime. In this specification the term “hydrocarbon conversion process” is therefore used generically to include processes such as Fluid Catalytic Cracking (FCC) and Resid Catalytic Cracking (RCC) in which a hydrocarbon provides the starting material and processes such as methanol conversion (which also includes dimethyl ether conversion) in which an organic feed stream such as methanol or a biofeed such as vegetable oil, animal oil, fish oil or oil of biosynthetic origin e.g. biosynthetic bacterial oil, is converted to a hydrocarbon product. The term “conversion” is used to mean any process by which one organic material is chemically and/or physically transmuted into another material with different chemical or physical properties. Thus, the term comprehends the physical and chemical changes in boiling point which occurs in fluid catalytic cracking where the average molecular weight is reduced in the process and in the methanol conversion processes such as methanol to olefins and aromatics and methanol alkylation of light aromatics where new species are produced.

The FIGURE shows how an air heater may be integrated into an FCCU in order to carry out startup without invoking the use of torch oil. The startup procedure is described here with reference to the FCCU as the epitome of the fluidized bed unit but, as noted below, the same technique may also be applied to other fluidized units requiring a high temperature startup.

The unit comprises a reaction section (not shown, as conventional) which is connected to a regenerator in the conventional manner by catalyst standpipes and/or transfer lines. The regenerator in the FIGURE is one of the dense bed types which the spent catalyst enters at a level part way up the regenerator vessel near the top of the dense fluidized bed and exits near the bottom (and vice versa) but the principle of initiating operation without use of torch oil would also be applicable to the riser-combustor type regenerator where the spent catalyst enters a combustor bulb at the bottom and exits near the top after passing up a riser to an upper bed. In the FIGURE, the unit 10 has a regenerator 11 which is fitted with an air grid 12 in which the regeneration gas, typically air or oxygen-enriched air is pumped from blower 13 by way of air heater 14; gas flow conduit 15 connects blower 13 to heater 14 and conduit 16 connects heater 14 to the air grid of regenerator 11. The catalyst inlet and outlet to and from regenerator 11 are not shown as conventional. Fuel for air heater 14 is supplied through inlet 18 and combusted in the heater with a conventional burner. Fuel gas is preferred as the fuel for the heater since it is readily available in refineries and petrochemical plants and has a relatively lower level of contaminants such as those commonly found in the oils used for torch oil, e.g. sulfur and metals. If lift gas is additionally required for process operation, for example, in FCC in the catalyst lift zone or in a catalyst riser in methanol conversion process as shown in U.S. Pat. No. 8,062,599, either heated lift gas or cold lift gas can be taken off from conduits 15 or 16 as required according to the heat demands for the operation in question.

While the equipment configuration is essentially conventional, its manner of operation represents a novel departure; instead of using torch oil to bring the regenerator to the required operating temperature after the initial dry out using the air heater, the air heater itself is used to bring the unit up to the temperature at which operation will be self-sustaining, if necessary with continued use of the air heater to maintain unit heat balance between the endothermic cracking reactions and the exothermic combustion in the regenerator. An exemplary startup sequence for an FCCU will be as follows:

1. The reactor and the refractory lining of the regenerator are dried out with hot air from the air heater; the spent/regenerated catalyst control valves in the standpipes lining the reactor and the regenerator are left open to permit circulation of the hot air.

2. The slide valves between the reactor and the regenerator are dosed and the reactor side is purged of oxygen with steam.

3. Steam is introduced to the reactor side through the lift gas injectors with pressure on the reactor side kept about 5-15 kPa (about 1-2 psi) higher than on the regenerator side.

4. Catalyst is loaded into the regenerator and heated up in parallel to the heating of the reactor to bring the regenerator up to same temperature as reactor 5. Transfer of catalyst from regenerator to reactor is initiated by opening the slide valves, and catalyst circulation on both sides is established.

5. The introduction of feed to reactor is started and the regenerator temperature increased to target temperature by use of the air heater.

Similar sequences will be followed for other fluidized catalytic process units with a reactor and regenerator regardless of whether the conversion reaction is endothermic or exothermic, for example, in the methanol conversion processes using a fluidized bed process unit. Processes of this type are known, including include the exemplary methylation of benzene/toluene with methanol or dimethyl ether to aromatics such as paraxylene, methanol conversion to olefins or aromatics or methanol conversion to gasoline. Exemplary methanol conversion processes, for example, include methylation of benzene and/or toluene as described in US 2013/0217940; U.S. Pat. Nos. 6,504,072; 6,642,426; methanol conversion to olefins and aromatics as described in U.S. Pat. No. 6,538,167 and conversion of methanol/DME to olefins, aromatics and non-aromatics, as described in U.S. Pat. No. 6,506,954. Other patents describing methanol conversion processes include U.S. Pat. No. 4,002,698; U.S. Pat. No. 4,356,338; U.S. Pat. No. 4,423,266; U.S. Pat. No. 5,675,047; U.S. Pat. No. 5,804,690; U.S. Pat. No. 5,939,597; U.S. Pat. No. 6,028,238; U.S. Pat. No. 6,046,372; U.S. Pat. No. 6,048,816; U.S. Pat. No. 6,156,949 and U.S. Pat. No. 6,423,879. In the case of the methanol conversion units an alternative way to heat up the catalyst without the use of torch oil, is from the reactor side. Here, a start-up heater (such as a toluene furnace) can be used with benzene, toluene, steam, N₂, H₂ or combinations of these being used as the heating medium to facilitate the startup sequence; when the circulating catalyst with the heating medium has reached a suitable temperature for the reaction to kick off, the feed of methanol or other alkylating agent can be initiated to start the reaction. If the aromatic substrate is used as the heating medium, it can be recycled from the fractionator of the product recovery section during the startup sequence.

The methanol conversion processes are markedly different from the FCC process in that the actual conversion reaction is strongly exothermic rather than endothermic; in addition, the catalyst circulation rate is much lower than in FCC with a lower catalyst:feed rate (about 10-12% wt as compared to a ratio of about 5:1 for FCC) and a lower catalyst circulation rate. This means that since the reaction itself is so strongly exothermic, the regenerator has a reduced work load (compared to the FCC regenerator) in supplying heat; the role of the regenerator in the methanol conversion units is therefore one of removing coke to restore catalytic activity and selectivity. The strongly exothermic nature of these processes, typically requires a catalyst cooler to carry off extraneous heat at some point in the cycle. Notwithstanding these differences, however, the process units require heating as a preliminary step in the startup and the use of the air heater to the exclusion of torch oil combustion for this purpose is advantageous for the same reasons as noted above.

To maintain overall thermal equilibrium, the methanol conversion units typically include a cooler which recycles partly coked catalyst back to the reaction section as described in U.S. Pat. No. 6,116,282; U.S. Pat. No. 8,062,599 and U.S. Pat. No. 7,084,319. Alternatively or in addition, the unit may be configured to pass some or all of the cooled catalyst into the stream of hot catalyst returning by way of the spent catalyst standpipe from the reactor and its associated stripper to the regenerator in order to control the temperature of the catalyst prior to entering the regenerator as described in US 2013/0165724; in a unit of this kind, it is advantageous for the combined catalyst stream (cooled catalyst plus hot, stripped catalyst) to enter the regenerator above the air grid and to this end, a vertically extended catalyst return riser may be provided, terminating at a higher level in the regenerator with hot or cold lift air supplied from gas flow conduits 15 or 16 according to the heat requirements of the process, entering at the bottom of the riser where the catalyst streams enter.

Depending on the composition of the feed streams used in methanol conversion units, for instance, methanol and/or dimethyl ether with benzene, toluene or other light aromatics, optionally with the addition of water, the startup sequence may need to be modified; in toluene alkylation with methanol, for example, the FCCU startup sequence described above might appropriately be adapted in step 6, by initiating feed introduction with the toluene and as a final step, to introduce the methanol feed stream.

In processes in which a large heat release takes place, e.g. in resid catalytic cracking, methanol conversion, where resort is made to catalyst coolers, the cooling function of the cooler should be disabled, for instance, by discontinuation of coolant flow during the startup sequence until sufficient coke is produced in the reactor and the regenerator temperature becomes high enough to maintain the desired reaction temperature.

Although the temperature of the circulating catalyst inventory will be less than the temperature of the air from the heater, the use of the heater should be adequate to raise the temperature to the required extent notwithstanding heat losses from the unit, assuming that the heater is adequately sized and that its metallurgy and that of the air transfer conduits is adequate to the required temperatures.

The present startup procedure is also suited in principle to other fluidized bed units without catalyst circulation but which require pre-heating to reaction temperature in order for the reaction to proceed and maintain itself. It is also applicable to non-catalytic fluidized bed units. The start-up procedure with methanol conversion units, for example, will follow the FCC procedure except that the reactor side may typically be purged of oxygen with a nitrogen loop established on the reactor side through the feed furnace and the product recovery section; when purging is complete, the system temperature is increased to the target temperature for the process. As noted above, the reactor may be brought up to reaction temperature by circulating heated catalyst from the regenerator or by supplying heat by way of the furnace on a reactor side feed stream.

In view of the expansion of the air in the heater when it is brought up to reaction temperature or close to it, some changes in the air handling components e.g. the air grid size and nozzle diameter and number, may become necessary in a revamped unit. These changes can be determined according to specific unit characteristics as needed.

The feed streams, catalysts and reaction conditions used in the processes will be selected according to the requirements of the process being operated in the unit and the type of unit in use. With FCC, for example, the feeds may be either distillate feeds such as gas oils, e.g. vacuum gas oil or residual feed such as vacuum resid; lighter co-feeds may be used along with the heavier oil. With methanol conversion processes, methanol and/or dimethyl ether will be used along with any other reactant, co-feed or promoter as well, optionally as hydrogen introduced into the reactor vessel. Exemplary reactants used with methanol or DME may include light aromatics such as benzene or toluene. In FCC, the catalysts will typically comprise a large pore size cracking component such as a faujasite zeolite, especially zeolite Y, REY or USY, commonly with an octane additive catalyst such as ZSM-5; the ZSM-5 additive catalysts are also useful for improved olefin production in catalytic cracking. In any event, once the process unit has been started using the air heater rather than torch oil, the process may be conducted within its normal operating envelope and subject to its normal constraints. Catalysts used in methanol conversion processes are typically intermediate pore size zeolites such as ZSM-5, ZSM-11 or MCM-22 or, alternatively, silicoaluminophosphates. Zeolites with a silica:alumina ratio of at least 250:1 and preferably about 500:1 are preferred.

1. A fluidized bed hydrocarbon conversion process in which a feed stream is converted in a fluidized bed process unit at an elevated temperature, comprising the step of starting up the unit by heating the unit to a self-sustaining reaction temperature with heated air from a heater.

2. A process according to claim 1 in which the unit is heated to a self-sustaining reaction temperature exclusively with heated air from an air heater.

3. A process according to claim 1 in which the unit is heated to a self-sustaining reaction temperature without burning hydrocarbon oil in the unit.

4. A process according to claim 1 in which the conversion process is an endothermic conversion process.

5. A process according to claim 4 in which the endothermic conversion process comprises Fluid Catalytic Cracking (FCC) of a heavy hydrocarbon feed.

6. A process according to claim 1 in which the conversion process is an exothermic conversion process.

7. A process according to claim 6 in which the conversion process comprises methanol conversion to aromatics or olefins.

8. A fluidized bed catalytic cracking process in which a heavy oil feed stream is catalytically cracked in a Fluid Catalytic Cracking (FCC) process unit at an elevated temperature, comprising the step of starting up the unit by heating the unit to a self-sustaining reaction temperature exclusively with heated air from an air heater.

9. A fluidized bed catalytic cracking process according to claim 8 in which the FCC process unit comprises a cracking reactor in which the heavy oil feed is cracked with a stream of hot catalyst from the catalyst regenerator in which the catalyst is regenerated by combustion of coke accumulated on the catalyst during the cracking of the heavy oil feed, the process including a unit startup in which heat is supplied to the cracking reactor and to the regenerator by the heated air from the air heater.

10. A fluidized bed catalytic cracking process according to claim 9 in which the air heater feeds heated air to the regenerator during the startup until the regenerator attains a temperature at which heat from the exothermic combustion of the coke accumulated on the catalyst during the cracking of the heavy oil feed is sufficient to sustain the cracking reaction.

11. A fluidized bed catalytic cracking process according to claim 10 in which cracking catalyst is loaded into the regenerator when the regenerator attains a temperature at which heat from the exothermic combustion of the coke accumulated on the catalyst during the cracking of the heavy oil feed is sufficient to sustain the cracking reaction.

12. A fluidized bed catalytic cracking process according to claim 11 in which the cracking catalyst comprises a large pore size zeolite of the faujasite.

13. A methanol conversion process in which a feed stream comprising methanol or dimethyl ether is converted in a fluidized bed methanol conversion process unit at an elevated temperature, comprising the step of starting up the unit by heating the unit to a self-sustaining reaction temperature exclusively with heated fluid from a heater.

14. A methanol conversion process according to claim 13 in which the methanol conversion process unit comprises a conversion reactor in which the feed is converted in the presence of a stream of catalyst from the catalyst regenerator in which the catalyst is regenerated by combustion of coke accumulated on the catalyst during the conversion of the feed, the process including a unit startup in which heat is supplied to the reactor and/or to the regenerator by heated air from an air heater.

15. A methanol conversion process according to claim 14 in which the air heater feeds heated air to the regenerator during the startup until the regenerator attains a temperature at which regenerated catalyst from which coke accumulated on the catalyst during the conversion has been combusted in the regenerator has sufficient conversion activity to sustain the conversion reaction.

16. A methanol conversion process according to claim 15 in which the catalyst is loaded into the regenerator when the regenerator attains the temperature at which the regenerated catalyst has sufficient conversion activity to sustain the conversion reaction.

17. A methanol conversion process according to claim 13 in which the feed is converted to olefins and aromatics in the conversion reaction.

18. A methanol conversion process according to claim 13 in which the feed comprises methanol and/or dimethyl ether and a light aromatic which is subjected to methylation in the conversion reaction to form an alkylated aromatic.

19. A methanol conversion process according to claim 18 in which the feed comprises methanol and/or dimethyl ether and toluene which is subjected to methylation in the conversion reaction to form xylene.

20. A methanol conversion process according to claim 18 in which the feed comprises methanol and/or dimethyl ether and toluene which is subjected to methylation in the conversion reaction to form paraxylene.

21. A methanol conversion process according to claim 13 in which the catalyst comprises ZSM-5.

22. A methanol conversion process according to claim 13 in which the catalyst comprises ZSM-5 having a silica:alumina ratio of at least 250:1.
